(19) United States
(12) Patent Application Publication
(10) Pub. No.: US 2022/0339192 A1
(43) Pub. Date: Oct. 27, 2022

(54) GENE EDITING FOR VIRAL INFECTIONS

(71) Applicant: THEMBA INC., SAN CARLOS, CA (US)

(72) Inventor: MATTHEW RABINOWITZ, SAN FRANCISCO, CA (US)

(73) Assignee: THEMBA INC., SAN CARLOS, CA (US)

(21) Appl. No.: 17/640,711

(22) PCT Filed: Sep. 4, 2020
(86) PCT No.: PCT/US2020/049424

§ 371 Date: Mar. 4, 2022

Related U.S. Application Data
(60) Provisional application No. 62/897,311, filed on Sep. 7, 2019.

(51) Int. Cl. A61K 35/17; A61P 31/12

(52) U.S. Cl. CPC A61K 35/17; A61P 31/12

(57) ABSTRACT

Provided are methods of treating a viral infection, the methods comprising administering, to a subject infected with a virus, a genetic modifying agent that targets one or more regions of the viral genome that are maximally different from the host genome. Also provided are methods comprising administering, to a subject infected with a virus, a genetic modifying agent that targets one or more genes of host cells that harbor the virus, where the targeted gene(s) are necessary to the correct function and replication of the virus but are dispensable to the host cells. Also provided are methods comprising decreasing, in one or more cells in the subject, the amount of susceptibility genetic variant(s); and/or increasing, in one or more cells in the subject, the amount of one or more protective genetic variant(s). Also provided are methods of identifying viral genes associated with survival of a virus.

### CROSS REFERENCE TO RELATED APPLICATIONS

This application claims the benefit of U.S. Provisional Application No. 62/897,311, filed on Sep. 7, 2019, which is incorporated herein by reference in its entirety.

### BACKGROUND

Treatment of viral infections (e.g., including Herpesviridae virus family such as Herpes Simplex Virus-1&2 (HSV-1, HSV-2), Epstein-Barr Virus (EBV), Human Immunodeficiency Virus (HIV), Hepatitis B Virus (HBV) and Human Papillomavirus (HPV)) is challenging for a variety of reasons associated with, e.g., viral life cycles, a lack of effective antiviral therapies, and the emergence of drug resistance to available antiviral therapies. Moreover, treatment of viral infections via gene editing techniques have been limited by lack of efficacy and/or delivery challenges. Provided are methods that address various shortcomings of currently available viral treatments.

### SUMMARY

Provided are methods of treating a viral infection, the methods comprising administering, to a subject infected with a virus, a genetic modifying agent that targets one or more regions of the viral genome that are maximally different from the subject's genome. In some aspects, the targeted region of the viral genome has less than 50% sequence homology with a region of the subject's genome, less than 40% sequence homology with a region of the subject's genome, less than 30% sequence homology with a region of the subject's genome, less than 20% sequence homology with a region of the subject's genome, less than 10% sequence homology with a region of the subject's genome, less than 5% sequence homology with a region of the subject's genome, or less than 1% sequence homology with a region of the subject's genome.

In some aspects, the genetic modifying agent is formulated in a carrier that has an increased affinity for cell types or tissue types harboring the viral infection as compared to cell types or tissue types not harboring the viral infection. In some aspects, the carrier comprises an adeno-associated virus vector, a lentiviral vector, or a polymer nanoparticle. In some aspects, the carrier comprises a polymer nanoparticle that comprises one or more of a cholesterol, a lipid-poly(ethylene glycol) (lipid-PEG) compound, an ionizable cationic lipid, a distearoylphosphatidylcholine, and a helper lipid (e.g., dioleoyl phosphatidylethanolamine (DOPE) and/or dioleoylphosphatidylcholine (DOPC)).

Also provided are methods of treating a viral infection, the methods comprising administering, to a subject infected with a virus, a genetic modifying agent that targets one or more genes of host cells that harbor the virus, where the targeted gene(s) are necessary to the correct function and replication of the virus but are dispensable to the host cells.

Also provided are methods of treating a viral infection, the methods comprising (a) decreasing, in one or more cells in the subject, the amount of one or more genetic variants associated with susceptibility to the viral infection (“susceptibility genetic variant(s)”); and/or (b) increasing, in one or more cells in the subject, the amount of one or more genetic variants protective against the viral infection (“protective genetic variant(s)”). Some aspects comprise decreasing the amount of the susceptibility genetic variant in one or more immune cells and/or one or more hematopoietic stem cells in the subject, and/or increasing the amount of the protective genetic variant in one or more immune cells and/or one or more hematopoietic stem cells in the subject. In some aspects, the immune cells comprise one or more of leukocytes, phagocytes, macrophages, neutrophils, dendritic cells, innate lymphoid cells, eosinophils, basophils, natural killer cells, B cells, and T cells.

Some aspects comprise administering to the subject: immune cells and/or hematopoietic stem cells containing the protective genetic variant; and/or immune cells and/or hematopoietic stem cells that contain the protective genetic variant and do not contain the susceptibility genetic variant. Some aspects comprise administering to the subject (i) a genetic modifying agent that decreases the amount of the susceptibility genetic variant in one or more immune cells and/or one or more hematopoietic stem cells in the subject, and/or (ii) a genetic modifying agent that increases the amount of the protective genetic variant in one or more immune cells and/or one or more hematopoietic stem cells in the subject. In some aspects, a proportion of protective protein variants:susceptibility protein variants in the subject is increased.

In some aspects, the genetic modifying agent comprises a nuclease. In some aspects, the nuclease is (1) a class 2 clustered regularly-interspaced short palindromic repeat (CRISPR) associated nuclease, (2) a zinc finger nuclease (ZFN), (3) a Transcription Activator-Like Effector nuclease (TALEN), or (4) a meganuclease. In some aspects, the nuclease comprises Cas1, Cas1B, Cas2, Cas3, Cas4, Cas5, Cash, Cas7, Cas8, Cas9, Cas10, Cpf1, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, or Csf4. In some aspects, the genetic modifying agent comprises a Cas9 protein and a guide RNA. In some aspects, the genetic modifying agent comprises one or both of CRISRP-Cas9 and a guide RNA. In some aspects, the genetic modifying agent is activated and/or deactivated using a chemical, biological, or optical input.

Also provided are methods of identifying viral genes associated with survival of a virus, the method comprising editing one or more viral genes and assessing the ability of the virus to replicate and survive with the edited genes. Some aspects comprise editing two, three, or more viral genes. In some aspects, the one or more viral genes are maximally different from a host genome.

In some aspects, the virus comprises HIV, HSV-1, or HSV-2.

### DETAILED DESCRIPTION

All documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

Unless defined otherwise, technical and scientific terms used herein have the same meaning as commonly understood by one of ordinary skill in the art to which this disclosure pertains. Definitions of common terms and techniques in molecular biology may be found in one or more of Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); Methods in Enzymology (1955) (Colowick ed.); PCR 2: A Practical Approach (1995) (M. J. MacPherson, B. D. Hames, and G. R. Taylor eds.): Antibodies, A Laboratory Manual (1988) (Harlow and Lane, eds.); Antibodies, A Laboratory Manual, 2^{nd} ed. (2013) (E. A. Greenfield ed.); Animal Cell Culture (1987) (R. I. Freshney, ed.); Benjamin Lewin, Genes IX (2008), The Encyclopedia of Molecular Biology (1994) (Kendew et al. eds.), Molecular Biology and Biotechnology: a Comprehensive Desk Reference (1995) (Meyers ed.); Singleton et al., Dictionary of Microbiology and Molecular Biology 2^{nd} ed. (1994) (Sainsbury ed.), Advanced Organic Chemistry Reactions, Mechanisms and Structure 4^{th} ed. (1992) (March ed.); and Transgenic Mouse Methods and Protocols, 2^{nd} edition (2011) (Hofker and Deursen eds.).

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less from the specified value, unless specifically identified to mean a separate variation, such as +1-5% or less, +/−1% or less, and +/−0.1% or less of and from the specified value. It is to be understood that the value to which the modifier “about” or “approximately” refers is itself also specifically, and preferably, disclosed.

The term “hematopoietic stem cells” or “HSCs” or “hematopoietic bone marrow stem cells” as used herein, refers to hematopoietic cells that are pluripotent stem cells or multipotent stem cells or lymphoid or myeloid (derived from bone marrow) cells that can differentiate into a hematopoietic progenitor cell (HPC) of a lymphoid, erythroid or myeloid cell lineage or proliferate as a stem cell population without initiation of further differentiation. HSCs can be obtained e.g., from bone marrow, peripheral blood, umbilical cord blood, amniotic fluid, or placental blood or embryonic stem cells. HSCs are capable of self-renewal and differentiating into or starting a pathway to becoming a mature blood cell e.g., erythrocytes (red blood cells), platelets, granulocytes (such as neutrophils, basophils and eosinophils), macrophages, B-lymphocytes, T-lymphocytes, and Natural killer cells through the process of hematopoiesis. The term “hematopoietic stem cells” encompasses “primitive hematopoietic stem cells” i.e., long-term hematopoietic stem cells (LT-HSCs), short-term hematopoietic stem cells (ST-HSCs) and multipotent progenitor cells (MPP).

The term “immune cell” as used herein generally encompasses any cell derived from a hematopoietic stem cell that plays a role in the immune response. Immune cells include, without limitation, lymphocytes, such as T cells and B cells, antigen-presenting cells (APC), dendritic cells, monocytes, macrophages, natural killer (NK) cells, mast cells, basophils, eosinophils, or neutrophils, as well as any progenitors of such cells. In certain preferred aspects, the immune cell may be a T cell. As used herein, the term “T cell” (i.e., T lymphocyte) is intended to include all cells within the T cell lineage, including thymocytes, immature T cells, mature T cells and the like. The term “T cell” may include CD4⁺ and/or CD8⁺ T cells, T helper (Tₕ) cells, e.g., Tₕ1, Tₕ2 and Tₕ17 cells, and T regulatory (T_{reg}) cells.

The term “modified” as used herein broadly denotes that an immune cell or HSC has been subjected to or manipulated by a man-made process, such as a man-made molecular or cell biology process, resulting in the modification of at least one characteristic of the cell. Such man-made process may for example be performed in vitro or in vivo.

The term “altered expression” denotes that the modification of the immune cell or HSC alters, i.e., changes or modulates, the expression of the recited gene(s) or polypeptides(s). The term “altered expression” encompasses any direction and any extent of said alteration. Hence, “altered expression” may reflect qualitative and/or quantitative change(s) of expression, and specifically encompasses both increase (e.g., activation or stimulation) or decrease (e.g., inhibition) of expression.

The terms “increased” or “increase” or “upregulated” or “upregulate” as used herein generally mean an increase by a statically significant amount. For avoidance of doubt, “increased” encompasses a statistically significant increase of at least 10% as compared to a reference level, including an increase of at least 20%, at least 30%, at least 40%, at least 50%, at least 60%, at least 70%, at least 80%, at least 90%, at least 100% or more, including, for example at least 2-fold, at least 3-fold, at least 4-fold, at least 5-fold, at least 10-fold increase or greater as compared to a reference level.

The term “reduced” or “reduce” or “decrease” or “decreased” or “downregulate” or “downregulated” as used herein generally means a decrease by a statistically significant amount relative to a reference. For avoidance of doubt, “reduced” encompasses statistically significant decrease of at least 10% as compared to a reference level, for example a decrease by at least 20%, at least 30%, at least 40%, at least 50%, at least 60%, at least 70%, at least 80%, at least 90%, or more, up to and including a 100% decrease (i.e., absent level as compared to a reference sample), or any decrease between 10-100% as compared to a reference level. The term “abolish” or “abolished” may in particular refer to a decrease by 100%, i.e., absent level as compared to a reference sample.

The terms “quantity”, “amount” and “level” are synonymous and generally well-understood in the art. The terms may particularly refer to an absolute quantification of a marker in a tested object (e.g., in or on a cell, cell population, tissue, organ, or organism, e.g., in a biological sample of a subject), or to a relative quantification of a marker in a tested object, i.e., relative to another value such as relative to a reference value, or to a range of values indicating a base-line of the marker. For example, the base-line or reference value can be obtained based on a determination of the quantity, level, or amount of a genetic variant in a subject (or in cells or populations of cells from the subject) having a viral infection or not having a viral infection, or in a subject (or in cells or populations of cells from the subject) at risk of developing a viral infection or not at risk of developing a viral infection. Such values or ranges may be obtained as conventionally known. In some cases, the quantity, amount, or level is a measured concentration. Amounts can be quantified using known techniques, such as PCR, UV absorption, calorimetry, fluorescence-based measurement, diphenylamine reaction methods, and others. See, e.g., Li, Analytical Biochemistry, 451: 18-24 (2014); Figueroa-Gonzalez, Oncol. Lett., 13(6): 3982-88 (2017); Psifidi, PLOSE ONE, 10(1): e0115960 (18 pages) (2015); Current Protocols in Protein Science (1996) (Coligan et al., eds.); and Current Protocols in Molecular Biology (2003) (Ausubel et al., eds.).

Any one or more of the several successive molecular mechanisms involved in the expression of a given gene or polypeptide may be targeted by the immune cell or HSC modification. Without limitation, these may include targeting the gene sequence (e.g., targeting the polypeptide-encoding, non-coding and/or regulatory portions of the gene sequence), the transcription of the gene into RNA, the polyadenylation and where applicable splicing and/or other post-transcriptional modifications of the RNA into mRNA, the localization of the mRNA into cell cytoplasm, where applicable other post-transcriptional modifications of the mRNA, the translation of the mRNA into a polypeptide chain, where applicable post-translational modifications of the polypeptide, and/or folding of the polypeptide chain into the mature conformation of the polypeptide. For compartmentalized polypeptides, such as secreted polypeptides and transmembrane polypeptides, this may further include targeting trafficking of the polypeptides, i.e., the cellular mechanism by which polypeptides are transported to the appropriate sub-cellular compartment or organelle, membrane, e.g. the plasma membrane, or outside the cell.

Hence, “altered expression” may particularly denote altered production of the recited gene products by the modified immune cell or HSC. As used herein, the term “gene product(s)” includes RNA transcribed from a gene (e.g., mRNA), or a polypeptide encoded by a gene or translated from RNA.

As used herein, the term “gene” refers to a nucleic acid comprising an open reading frame encoding a polypeptide, including both exon and (optionally) intron sequences. A “gene” refers to the coding sequence of a gene product, as well as non-coding regions of the gene product, including 5′UTR and 3′UTR regions, introns and the promoter of the gene product. The coding region of a gene can be a nucleotide sequence coding for an amino acid sequence or a functional RNA, such as tRNA, rRNA, catalytic RNA, siRNA, miRNA and antisense RNA. A gene can also be an mRNA or cDNA corresponding to the coding regions (e.g. exons and miRNA) optionally comprising 5′ or 3′ untranslated sequences linked thereto. A nucleic acid may encompass a single-stranded molecule or a double-stranded molecule that comprises one or more complementary strand(s) or “complement(s)” of a particular sequence comprising a molecule. As used herein, a single-stranded nucleic acid may be denoted by the prefix “ss”, and a double stranded nucleic acid by the prefix “ds”. The term “gene” may refer to the segment of DNA involved in producing a polypeptide chain, and it includes regions preceding and following the coding region as well as intervening sequences (introns and non-translated sequences, e.g., 5′- and 3′-untranslated sequences and regulatory sequences) between individual coding segments (exons). A gene can also be an amplified nucleic acid molecule produced in vitro comprising all or a part of the coding region and/or 5′- or 3′-untranslated sequences linked thereto. The term “genetic variant” is used to refer to a version of a gene, such as a wildtype version, a mutated version, or a single-nucleotide polymorphism version. Some gene variants may be associated with increased susceptibility to a viral infection. Some gene variants may be protective against a viral infection.

The term “nuclease” as used herein broadly refers to an agent, for example a protein or a small molecule, capable of cleaving a phosphodiester bond connecting nucleotide residues in a nucleic acid molecule. In some aspects, a nuclease may be a protein, e.g., an enzyme that can bind a nucleic acid molecule and cleave a phosphodiester bond connecting nucleotide residues within the nucleic acid molecule. A nuclease may be an endonuclease, cleaving a phosphodiester bonds within a polynucleotide chain, or an exonuclease, cleaving a phosphodiester bond at the end of the polynucleotide chain. Preferably, the nuclease is an endonuclease. Preferably, the nuclease is a site-specific nuclease, binding and/or cleaving a specific phosphodiester bond within a specific nucleotide sequence, which may be referred to as “recognition sequence”, “nuclease target site”, or “target site”. In some aspects, a nuclease may recognize a single stranded target site, in other aspects a nuclease may recognize a double-stranded target site, for example a double-stranded DNA target site. Some endonucleases cut a double-stranded nucleic acid target site symmetrically, i.e., cutting both strands at the same position so that the ends comprise base-paired nucleotides, also known as blunt ends. Other endonucleases cut a double-stranded nucleic acid target sites asymmetrically, i.e., cutting each strand at a different position so that the ends comprise unpaired nucleotides. Unpaired nucleotides at the end of a double-stranded DNA molecule are also referred to as “overhangs”, e.g., “5′-overhang” or “3′-overhang”, depending on whether the unpaired nucleotide(s) form(s) the 5′ or the 3′ end of the respective DNA strand.

Provided are methods of treating viral infections. While it is believed that millions of viruses may exist, several thousand types have been identified to date. Without being bound by theory viruses are believed to be small parasites that cannot reproduce by themselves. Once it infects a susceptible cell, however, a virus can direct the cell machinery to produce more viruses. Most viruses have either RNA or DNA as their genetic material, which may be single- or double-stranded. The entire infectious virus particle, called a virion, consists of the nucleic acid and an outer shell of protein. The simplest viruses contain only enough RNA or DNA to encode four proteins. The most complex can encode 100-200 proteins or more.

Exemplary viruses treatable in accordance with the provided methods include Herpes Simplex viruses (HSV). HSV is discussed in greater detail in Groves, “Genital Herpes: A Review,” Am. Fam. Physician, 93(11): 928-934 (2016), which is incorporated herein by reference in its entirety. HSV infects about 70% of the U.S. population. HSV-1 most commonly initially infects the oral mucosal epithelium, leading to a local productive infection, and then undergoes retrograde transport to the trigeminal ganglia, where it establishes a latent infection in a small number of sensory neurons that persists after the initial, productive infection is cleared by the host immune response. During latency, the HSV-1 DNA genome is maintained as a nuclear episome, with from 1 to 50 copies per latently infected neuron. At this point, the only region of the genome that is actively transcribed encodes the latency associated transcript LAT, which is processed to give rise to a single long non-coding RNA of 2.1 kb, as well as 8 virally encoded miRNAs, that together are thought to regulate exit from latency. Importantly, no viral proteins are made, thus preventing immune recognition of latently infected cells. Occasionally, generally after some form of stress, one or more latently infected neuron is activated to produce infectious virions that migrate down the axons of the reactivating neuron to the original site of infection, where they re-establish a transient productive infection that can lead to the formation of cold sores. HSV-2 is found in about 20% of the US population and has a similar replication cycle but generally is sexually transmitted and often infects the genital mucosa. In the case of HSV-2, latency is established in sensory neurons of the sacral ganglia and reactivation can lead to genital ulcers. While there are several drugs that can treat productive HSV-1 or HSV-2 infections, generally by targeting viral DNA synthesis, latent HSV genomes are refractory to current treatment regimens and it remains impossible to cure these infections. Provided are therapeutic approaches that directly target HSV-1 or HSV-2 episomal DNA for cleavage and elimination from latently infected neurons.

Other exemplary viruses treatable with the provided methods include HIV. HIV is discussed in greater detail in Seitz, “Human Immunodeficiency Virus (HIV),” Transfus. Med. Hemother. 43(3): 203-222 (2016), which is incorporated herein by reference in its entirety. While highly active antiretroviral therapy (HAART) can reduce HIV-1 replication to levels below the detection limit, HIV-1 persists as a latent infection in a small number of resting CD4+ memory T cells. In these long lived cells, intact integrated HIV-1 proviruses persist in a transcriptionally silent state that is refractory to both drugs and host immune responses. However, these memory T cells can be reactivated by an appropriate recall antigen, resulting in induction of a productive viral replication cycle. If this occurs after drug treatment has been stopped, HIV-1 will rapidly spread through the available CD4+ T cells and rekindle the same level of virus replication that was seen prior to antiviral drug treatment. Efforts to purge the pool of latently infected cells have focused on two strategies. On the one hand, several groups have attempted to activate latent HIV-1 proviruses using drugs, including histone deacetylase inhibitors and PKC agonists (Xing and Siliciano, 2013). However, so far this strategy has not proven able to activate HIV-1 in a high percentage of latently infected cells.

Provided are strategies to directly target and destroy latent proviruses using HIV-1-specific CRISPR/Cas combinations. In principle, the HIV-1 provirus is a suitable target for CRISPR/Cas as there is only a single proviral copy in the infected cell and, in the presence of antiviral drugs, no spread of the virus is possible; however, latently HIV-1 infected T cells are scattered throughout the body thus hindering such an approach, especially as T cells are poor targets for AAV infection. This contrasts with HBV, HSV and HPV, all of which are tightly localized in known tissues in the body that can be readily targeted by AAV. Thus, provided are vector delivery systems that can target latently HIV-1 infected cells throughout the body.

### Editing Viruses

Provided are methods for targeting viral genomes for editing to destroy virus while posing minimal risk to host. Some aspects comprise using viral DNA targeted CRISPR/Cas9 gene editing constructs to target with sgRNA and excise a particular gene or genes crucial to the function of viruses, such as HSV or HIV virus.

### Delivery Mechanisms.

Some aspects comprise administering a genetic therapy to a subject (e.g., for editing of CD4+ cells). In some aspects, the genetic therapy is administered (e.g., intravenously, intramuscularly, or via other known administration routes) using a lentivirus or AAV carrier. In some aspects, the genetic therapy is administered using polymer nanoparticles. Without being bound by theory, it is believed that such nanoparticles have high tropism for T-cells and provide targeting, payload size capabilities, and manufacturing advantages as compared to AAV or lentiviral delivery systems, such as those in Kaminski, “Elimination of HIV-1 Genomes from Human T-lymphoid Cells by CRISPR/Cas9 Gene Editing,” Nature Scientific Reports, 6: Article No. 22555 (2016). For instance, polymer nanoparticles can have high tropism for T-cells, can carry DNA packages of sufficient size and can be manufactured to have orders of magnitude higher infectious units per ml than lentiviruses.

Targeting Regions Maximally Differentiated from Host Genome.

Some aspects comprise performing a genome sequence search to identify regions in the viral genome that are maximally different from the host genome. Some aspects comprise using targeting (e.g. with sgRNA) viral genes that are unlikely to target the host genome. Such an approach yields improved efficacy as compared targeting the multiple repeat regions of the viral genome, e.g., as mentioned in Wang, RNA-guided endonuclease provides a therapeutic strategy to cure latent herpesviridae infection,” Proc. Natl. Acad. Sci. USA, 111(36): 13157-62 (2014). Some aspects comprise administering to a subject a genetic modifying agent that targets a region in the viral genome that has less than 50% sequence homology with a region (e.g., a corresponding region or any region) of the host genome, such as less than 40% sequence homology, less than 30% sequence homology, less than 20% sequence homology, less than 10% sequence homology, less than 5% sequence homology, or less than 1% sequence homology. Some aspects comprise administering to a subject a genetic modifying agent that targets a region in the viral genome that has less than 50% sequence identity with a region of the host genome, such as less than 40% sequence identity, less than 30% sequence identity, less than 20% sequence identity, less than 10% sequence identity, less than 5% sequence identity, or less than 1% sequence identity.

Identifying Key Genes where Incision Destroys Virus' Ability to Repair and Replicate.

Some aspects comprise identifying genes necessary for viral repair and replication. In some aspects, the identification is performed using in-vitro experiments. Some aspects comprise editing genes singly, in pairs, in triplets, or more and analyzing viral ability to survive and replicate. Such an improve would provide an improvement over the results of Wang 2014, where double excision of EBNA1 (EBV Nuclear Antigen gene) showed little effect. Exemplary genes or proteins that could be effective targets on HIV include the HIV-1 Vif and Vpu proteins. The HIV-1 Vif protein blocks the activity of the host APOBEC3 family of restriction factors, which otherwise interfere with the production of the HIV-1 provirus. The HIV-1 Vpu protein neutralizes the cellular restriction factor tetherin, which otherwise blocks the release of progeny virions. Other exemplary gene targets for HIV-1 are disclosed in Li, “HIV Genome-Wide Protein Associations: a Review of 30 Years of Research,” Microbiol. Mol. Biol. Rev., 80(3): 679-731 (2016). Exemplary genes targets for HSV2 are disclosed in Jiao et al, “Complete Genome Sequence of Herpes Simplex Virus 1 Strain McKrae,” Microbiology Resource Announcements, 8(39): e00993-19 (2019), which is incorporated herein by reference in its entirety. Exemplary genes targets for HSV1 are disclosed in Dolan et al, “The Genome Sequence of Herpes Simplex Virus Type 2,” Journal of Virology, 72(3): 2010-2021 (1998), which is incorporated herein by reference in its entirety.

Targeting Delivery of CRISPR/Cas9 to Particular Cell Types In-Vivo that Harbor the Virus.

Some aspects comprise testing various types of AAVs, lentiviruses, and/or polymer nanoparticles for their affinity to the particular cell types that harbor the virus, such CD4+ T cells for HIV, dorsal or sacral nerve ganglia in the case of HSV, and various other cell types for a host of other viruses. Some aspects (e.g., in the case of using polymer of lipid delivery vectors) comprise using a unique barcode each subset of delivery agent (e.g., each nanoparticle type) and then delivering a large number of nanoparticles to a subject (e.g., a test animal). Some aspects comprise sacrificing the animal (which can be a single animal) to determine which particle types are found in which tissue. For example, lipid nanoparticle libraries can be generated by combining different biomaterials with cholesterols, lipid-poly(ethylene glycol) (lipid-PEG) compounds, ionizable cationic lipids, distearoylphosphatidylcholine, and helper lipids (e.g., dioleoyl phosphatidylethanolamine (DOPE); dioleoylphosphatidylcholine (DOPC)). Some aspects comprise generating nanoparticle libraries by varying the molar ratio of biomaterials, cholesterols, PEG, and helper lipids. In some aspects, between 10⁸ and 2.10¹¹ chemically distinct nanoparticles are made by combining these compounds. The size of the libraries can be further increased by varying the molar ratio of the compounds. High-throughput nanoparticle formulation allows for the rapid production of large, diverse libraries, of lipid, polymer and other nanoparticles with each nanoparticle type uniquely DNA barcoded. See for example Lokugamage et al., Testing thousands of nanoparticles in vivo using DNA barcodes, Current Opinion in Biomedical Engineering, 7: 1-8 (2018), which is incorporated herein by reference in its entirety.

Some aspects comprise testing whether a viral load is reduced by particular delivery vectors. For HSV virus in particular, several AAV serotypes, including AAV8, are able to infect almost all of the sensory neurons in the dorsal root ganglion after application of particles of a green fluorescent protein (gfp) expressing AAV8 vector to tissue of the mouse in vivo, such as the rear footpad. Latent HSV-1 infections of neurons in the mouse trigeminal ganglia (TGs) can be readily established and it is therefore possible to test whether transduction of these same TGs with AAV8-based vectors encoding HSV-1-specific Cas9/gRNA combinations will result in a detectable reduction in viral DNA load and an inhibition in the ability of latent HSV-1 to reactivate after explant and culture of the infected TGs.

### Delivery of CAS9 Protein Instead of DNA.

Some aspects provide delivering, alone or in combination with the approaches above, a Cas9 protein rather than DNA. In some aspects, such an approach has superior specificity and safety since the DNA is not continually replicated to produce new Cas9 enzyme. As mentioned, polymer nanoparticles is one possible approach to delivery of Cas9 protein along with the gRNA.

### Improved Safety Mechanisms.

Previous approaches (e.g. Wang et al 2014) included in the plasmid that contained the Cas9 sequence, the OriP or EBV origin of replication sequence. This was likely necessary in order to implement a safety mechanism so that off-target gene editing would not continue once the viral load had reduced. The disadvantage of this method is that it causes negative feedback and becomes less efficient as the viral load diminishes. Indeed, Wang et al. only achieve 85% reduction of viral load, possibly because of this in-built negative feedback. Thus, some aspects provided comprise switches that do not suffer from this negative feedback mechanism. That is, some aspects comprise switching the activity of the Cas9 protein for excising viral DNA using chemical or optical inputs. Some embodiments comprise using the methods described, for example, in: Davis et al., “Small molecule-triggered Cas9 protein with improved genome-editing specificity,” Nat. Chem. Biol., 11: 316-318 (2015); Liu, et al., “A chemical-inducible CRISPR-Cas9 system for rapid control of genome editing,” Nat. Chem. Biol., 12: 980-987 (2016); Maji et al., Multidimensional chemical control of CRISPR-Cas9, Nat. Chem. Biol., 13: 9-11 (2017); Nguyen et al., “Ligand-binding domains of nuclear receptors facilitate tight control of split CRISPR activity,” Nat. Commun. 7: 12009 (2016); Oakes et al., “Profiling of engineering hotspots identifies an allosteric CRISPR-Cas9 switch,” Nat. Biotechnol., 34: 646-651 (2016); Zetsche, et al., “A split-Cas9 architecture for inducible genome editing and transcription modulation,” Nat. Biotechnol., 33: 139-142 (2015); Gao, et al., “Complex transcriptional modulation with orthogonal an inducible dCas9 regulators,” Nat. Methods 13: 1043-1049 (2016); Hemphill, et al., “Optical control of CRISPR/Cas9 gene editing,” J. Am. Chem. Soc., 137: 5642-5645 (2015); Nihongaki, et al., “M. Photoactivatable CRISPRCas9 for optogenetic genome editing,” Nat. Biotechnol., 33: 755-760 (2015); and/or Polstein, et al. “A light-inducible CRISPR-Cas9 system for control of endogenous gene activation,” Nat. Chem. Biol, 11: 198-200 (2015), each of which are herein incorporated by reference in their entireties.

Some embodiments comprise targeting multiple genes that are regulated in the same manner. Some embodiments comprise targeting genes with orthogonal CRISPR-Cas9 systems to regulate different genes independently. Some embodiments comprise regulating the sgRNA instead of the Cas9 protein. Since the sgRNA is specific for each target sequence, controlling the sgRNA directly can independently regulate each target. Some embodiments comprise using sgRNAs that sequesters a 20 nucleotide target sequence only in the absence of an RNA binding ligand. Some embodiments comprise using the methods described, for example in: Liu, et al., “Directing cellular information flow via CRISPR signal conductors,” Nat. Methods, 13: 938-944 (2016); and/or Tang, et al., “Aptazyme-embedded guide RNAs enable ligand-responsive genome editing and transcriptional activation,” Nat. Commun., 8: 15939 (2017), both of which are incorporated herein by reference in their entirety.

Some embodiments comprise implementing direct and gene-specific control of sgRNA by ligand-dependent ribozymes that cause irreversible RNA cleavage. Such methods are described for sample in Tang, et al., “Aptazyme-embedded guide RNAs enable ligand-responsive genome editing and transcriptional activation,” Nat. Commun., 8: 15939 (2017) and/or Ferry, et al., “Rational design of inducible CRISPR guide RNAs for de novo assembly of transcriptional programs,” Nat. Commun., 8: 14633 (2017), both of which are incorporated herein by reference in their entirety.

Direct and gene-specific control of the sgRNA can further be implemented using ligand-dependent protein regulators recruited to the sgRNA to alter CRISPR function, as in Maji et al., 2017, or by engineered antisense RNA to sequester and inactivate the sgRNA—see for example: Lee, et al. Programmable control of bacterial gene expression with the combined CRISPR and antisense RNA system. Nucleic Acids Res. 44, 2462-2473 (2016).

Some aspects comprise using of RNA aptamers, such as theophylline aptamers, to directly affect functional interactions between the sgRNA, Cas9, and the DNA target. Such an approach can be used to activate and/or deactivate CRISPR-Cas9 function in response to a small molecule that can be separately dosed, with activation and deactivation scaling with dosage over a wide dynamic range. Such methods are described, for example in Kundert et al., “Controlling CRISPR-Cas9 with ligand-activated and ligand-deactivated sgRNAs,” Nat. Commun., 10: 2127 (2019), which is incorporated herein by reference in its entirety. Kundert showed the activation or deactivation of CRISPR-Cas9-based gene repression in a dose-dependent fashion over a >10-fold dynamic range in response to two different small-molecule ligands. This system acts directly on each target-specific sgRNA. Note that Theophylline (as described in Kundert) is a drug already used in therapy for respiratory diseases such as chronic obstructive pulmonary disease (COPD) and asthma under a variety of brand names.

Some aspects comprise limiting the activity of CRISPR/Cas-9 in the context of viral editing by one or more of: delivering Cas9 protein in two parts, with a separate molecule delivered to combine parts and activate enzyme; separate dosing of peptides known to restrict activity of Cas9; and targeting different non-viral replication sites on the plasmid or other DNA containing Cas9, unique to an enzyme who's levels can be separately controlled as with “biologic” drugs. Many other switches are possible that control either replication of the DNA coding for the CAS9 protein, or the editing of the viral genome by the Cas9.

### Editing Host Genes

Also provided are methods of identifying and/or editing host genes. Some aspects comprise editing one or more genes of host cells that harbor the virus, where the targeted genes are necessary to the correct function and replication of the virus but dispensable to the host. Some aspects comprise conducting a screening method to identify genes that undermine viral survival. Exemplary screening methods can include steps set forth in Shalem et al., “Genome-scale CRISPR-Cas9 knockout screening in human cells,” Science, 343(6166): 84-87 (2014); Wang et al., “Genetic screens in human cells using the CRISPR-Cas9 System,” Science, 343(6166): 80-84 (2014); and/or Zhou et al., “High-throughput screening of a CRISPR/Cas9 library for functional genomics in human cells,” Nature, 509: 487-491 (2014), each of which are incorporated herein by reference in their entireties.

Some aspects comprise editing hematopoietic bone marrow stem cells to include variants that are protective to viruses, such as HIV. In some aspects, the hematopoietic bone marrow stem cells then make leukocytes, such as T-cells, that are resistant to infection.

Some aspects comprise methods of decreasing, in one or more cells in a subject, the amount of one or more genetic variants associated with susceptibility to a viral infection; and/or increasing, in one or more cells in the subject, the amount of one or more genetic variants protective against or resistant to the viral infection. In some aspects, the methods comprise decreasing the amount of the susceptibility genetic variant in one or more immune cells and/or one or more hematopoietic stem cells in the subject, and increasing the amount of the protective genetic variant in one or more immune cells and/or one or more hematopoietic stem cells in the subject.

In some aspects the cells are immune cells and/or hematopoietic stem cells. In some aspects, the immune cells comprise one or more of leukocytes, phagocytes, macrophages, neutrophils, dendritic cells, innate lymphoid cells, eosinophils, basophils, natural killer cells, B cells, and T cells.

Some aspects comprise administering to the subject immune cells and/or hematopoietic stem cells containing the protective genetic variant; and/or immune cells and/or hematopoietic stem cells that contain the protective genetic variant and do not contain the susceptibility genetic variant. In some aspects, the proportion of protective protein variants:susceptibility protein variants in the subject (or in cells or a population of cells in the subject) is increased. That is, the amount of protective protein variants is increased relative to the amount of susceptibility protein variants.

Some aspects comprise obtaining immune cells and/or hematopoietic stem cells from a first subject, altering the obtained immune cells and/or hematopoietic stem cells to decrease the amount of the susceptibility genetic variant and/or increase the amount of the protective genetic variant, and administering the altered immune cells and/or hematopoietic stem cells to the subject in need of treatment. In some aspects, the immune cells and/or hematopoietic stem cells are obtained from the subject's blood or bone marrow. In some aspects, the first subject is the subject in need of treatment. In some aspects, the altered immune cells and/or hematopoietic stem cells are administered via venous administration or via a bone marrow transplant.

Some aspects comprise eliminating at least a portion of the hematopoietic stem cells in the subject prior to administration of the immune cells and/or hematopoietic stem cells. In some aspects, the eliminating comprises administering chemotherapy or radiation to the subject; administering anti-c-Kit monoclonal antibodies to the subject; and/or administering a CD47 blockade to the subject.

Some aspects comprise administering a genetic modifying agent to the subject, wherein the genetic modifying agent (a) decreases the amount of the susceptibility genetic variant in one or more cells in the subject, and/or (b) increases the amount of the protective genetic variant in one or more cells in the subject. In some aspects, the genetic modifying agent comprises a nuclease. In some aspects, the nuclease is (1) a class 2 clustered regularly-interspaced short palindromic repeat (CRISPR) associated nuclease, (2) a zinc finger nuclease (ZFN), (3) a Transcription Activator-Like Effector nuclease (TALEN), or (4) a meganuclease. For example, in some aspects the nuclease comprises Cas1, Cas1B, Cas2, Cas3, Cas4, Cas5, Cash, Cas7, Cas8, Cas9, Cas10, Cpf1, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, or Csf4. In some aspects, the nuclease comprises Cas9 or Cpf1.

Also provided are methods of treating a subject having an viral infection, the method comprising editing DNA in immune cells and/or hematopoietic stem cells in a subject to: (a) decrease the amount of one or more genetic variants associated with (i) resistance to a particular drug for treating the viral infection or (ii) a distribution of bacteria in the bowel of the subject associated with increased susceptibility to the viral infection; and/or (b) increase the amount of one or more genetic variants associated with (i) increased sensitivity to a particular drug for treating the viral infection or (ii) a distribution of bacterial in the bowel of a subject that is protective of the viral infection.

In some aspects, the susceptibility genetic variant and the protective genetic variant are determined based on one or more of: (a) the phenotype of one or more family members, sequencing a panel of genes in one or more family members, whole exome sequencing in one or more family members, and/or whole genome sequencing of one or more family members; (b) computer simulations of cellular signaling and/or response to viral infection; (c) machine modeling of mutations that affect phenotypes, such as with linear and/or nonlinear regression models, neural networks; (d) data describing gene expression and/or gene signaling; and (e) animal models (e.g., pigs).

Also provided are isolated immune cells or hematopoietic stem cell, wherein the cellular DNA has been modified via gene editing to (a) reduce the amount of one or more genetic variants associated with susceptibility to a viral infection; and/or (b) increase the amount of one or more genetic variants protective against the viral infection.

Also provided are populations of immune cells or hematopoietic stem cells, wherein at least about 10% of the cells in the population have been modified via gene editing to (a) reduce the amount of one or more genetic variants associated with susceptibility to a viral infection; and (b) increase the amount of one or more genetic variants protective against the viral infection.

In some aspects, the compositions comprise (i) a nucleic acid encoding a CRISPR/Cas nuclease, (ii) a guide RNA, or a nucleic acid encoding the guide RNA, that hybridizes to a target sequence within the genomic DNA of the cell that encodes a genetic variant not protective of a viral infection, and (iii) a DNA repair template encoding a genetic variant protective of a viral infection at the location of the target sequence.

In some aspects, the compositions comprise (a) (i) a nucleic acid encoding a CRISPR/Cas nuclease, (ii) a guide RNA, or a nucleic acid encoding the guide RNA, that hybridizes to a target sequence within the genomic DNA of the cell that encodes a genetic variant associated with susceptibility to viral infection, and (iii) a DNA repair template encoding a genetic variant not associated with susceptibility to an viral infection; and (b) (i) a nucleic acid encoding a CRISPR/Cas nuclease, (ii) a guide RNA, or a nucleic acid encoding the guide RNA, that hybridizes to a target sequence within the genomic DNA of the cell that encodes a genetic variant not protective of a viral infection, and (iii) a DNA repair template encoding a genetic variant protective of a viral infection at the location of the target sequence.

In some aspects, the compositions comprise two or more guide RNAs, wherein the guide RNAs collectively hybridize to more than one target sequence.

### Target Population

Provided are methods and compositions for treating or preventing viral infection in a subject. The subject can be any animal, such as a mammalian animal (preferably a human). In some aspects, the subject has been diagnosed with a viral infection. In some aspects, the subject has or is at risk of developing a viral infection.

In some aspects, the subject is genetically susceptible to a viral infection. For instance, in some aspects the subject has one or more alleles associated with increased risk of having or developing a viral infection (i.e., a susceptibility genetic variant). Such an increased susceptibility to a viral infection can result in the subject having an increased amount of one or more protein variants associated with a viral infection (i.e., a susceptibility protein variant) as compared to a subject that does not have or is not at increased risk of developing the viral infection.

Susceptibility genes are not limited to genes that directly impact on development of the viral infection. For instance, in some aspects the susceptibility gene is associated with the subject's response to a particular therapy. In some aspects the susceptibility gene is associated with the subject's microbiome (e.g., gut microbiome), which without being bound by theory is believed to impact the development and progression of viral infection.

Susceptibility genes can be identified by any means, including means known in the art. For instance, in some aspects the susceptibility gene is known in the art to be associated directly or indirectly with a viral infection. In some aspects, the susceptibility gene is identified based on a family tree that includes relatives displaying or being affected by a particular phenotype. In some aspects, the susceptibility gene is identified via whole exome or whole genome sequencing of one or more family members. In some aspects, the susceptibility gene is identified by machine modeling, such as with neural networks or other linear and nonlinear regression models and/or using gene signaling networks where particular mutations are seen to disrupt gene signaling. In some aspects, the susceptibility gene is identified using animal models (e.g., pigs or mice). In some aspects, various methods of identifying susceptibility genes (e.g., one or more of identifying known susceptibility genes, identifying particular phenotypes with reference to a family tree, and whole exome or whole genome analysis) are combined to identify an individual in need to treatment. That is, in some aspects genes common to family members having susceptibility to viral infection are determined to be susceptibility genes.

### Methods of Treatment

Some aspects comprise methods and compositions that change the paradigm for treating viral infection. Some aspects comprise editing the genes of host cells that harbor the virus, where the targeted genes are necessary to the correct function and replication of the virus but dispensable to the host. Some aspects comprise editing the genes of host cells that harbor the virus, where the targeted genes are necessary to the correct function and replication of the virus but dispensable to the host.

Some aspects involve the use of gene editing to edit the genes of the immune cells, or the tissue that generates the immune cells by hematopoiesis. By changing the underlying genes to eliminate risk alleles, and/or create protective alleles, the complex gene signaling pathways do not need to be precisely understood in order to eliminate or reduce the severity of the viral infection.

In some aspects, the subject is administered a therapy that decreases the amount of one or more protein variants associated with susceptibility to viral infection. Such a decrease can occur in a subject or in one or more cells in the subject. In some aspects, the number of cells with one or more susceptibility genetic variants is decreased in the subject. For instance, in some aspects the subject is administered a therapy (e.g., a genetic modifying agent) that modifies a genetic variant in vivo and/or that decreases expression of a susceptibility protein variant. In some aspects, the therapy targets the susceptibility gene, e.g., with a gene editing or gene silencing technology. Some aspects involve targeting one or more of the susceptibility genes (including without limitation environmentally-mediated susceptibility genes, susceptibility genes that affect medication response, and susceptibility genes that affect management of the viral infection), and genes that interact with the susceptibility gene.

In some aspects, the methods comprise increasing the amount of one or more genetic variants protective against the viral infection. Such an increase can occur in a subject or in one or more cells in the subject. In some aspects, the number of cells with one or more protective genetic variants is increased in the subject. For instance, in some aspects the proportion of expression of protective protein variants is increased in the subject. In some aspects, the susceptibility gene is mutated via a genetic modifying agent to a wild type variant. In some aspects the susceptibility gene is mutated to a gene that is protective against the viral infection (i.e., a protective gene). In some aspects, a genetic variant that is not protective (e.g., a wildtype genetic variant that is not protective) is mutated to a gene that is protective against the viral infection.

In some aspects, the susceptibility gene and the protective gene encode variants of the same protein. In some aspects, the susceptibility gene and the protective gene encode variants of different proteins.

In some aspects, the subject is administered an agent that targets one or more susceptibility genetic variants and/or one or more protective genetic variants in immune cells and/or hematopoietic bone marrow stem cells. In some aspects, the subject is administered a genetic modifying agent to decrease the amount of susceptibility genetic variant in the subject and/or to increase the amount of protective protein variant in the subject. In some aspects, the subject is administered immune cells and/or HSCs that have been modified to decrease the amount of susceptibility genetic variant and/or increase the amount of protective genetic variant.

In some aspects, the proportion of protective genetic variant:susceptibility genetic variant is increased in the subject, or in cells or populations of cells in the subject. In some aspects, the ratio of protective protein variants:susceptibility protein variants is increased in the subject, or in cells or populations of cells in the subject.

In some aspects, HSC cells are harvested from the blood or bone marrow, and then a genetic modifying agent is used on the harvested cells ex vivo. In some aspects CD34+ cells from blood samples are isolated using immunomagnetic or immunofluorescent methods (The CD34 protein is a member of a family of single-pass transmembrane sialomucin proteins expressed on early hematopoietic and vascular-associated tissue. It is a cell surface glycoprotein and functions as a cell-cell adhesion factor and may mediate the attachment of hematopoietic stem cells to bone marrow extracellular matrix or directly to stromal cells). Although CD34+ is ubiquitously associated with HSCs, it is also found on other cell types. See for example Sidney, Stem Cells, 32(6): 1380-9 (2014). Clinically, it can be used for the selection and enrichment of hematopoietic stem cells for bone marrow transplants. CD34+ cells can be harvested from the blood using antibodies that bind to CD34 and are attached to magnetic beads or fluorescent markers to enable subsequent isolation and subsequent gene editing of these cells. These cells may also be cultured before or after gene editing. The cells may then be returned to the subject by blood transfusion or injection. In some aspects, the subject is subjected to chemotherapy or radiation to eliminate a significant portion of their bone marrow HSCs before the edited HSCs or CD34+ cells are returned to the blood, so that the bone marrow is recolonized with a significant portion of edited HSCs. In some aspects, this application uses a lower dosage of radiation or chemotherapy than the lethal dosages used for bone marrow cancer patients (e.g., to deplete the host HSCs, but not to eliminate the entire HSC population). In some aspects, prior to transfusion of the edited HSCs, antibodies that disrupt function of HSC and cause host HSC depletion are administered to the host. For example, HSCs express c-Kit (CD117), a dimeric transmembrane receptor tyrosine kinase, which is implicated in HSC function. Anti-c-Kit monoclonal antibodies can be used along with immune suppression to deplete HSCs from bone marrow niches, allowing edited HSC engraftment. In some aspects, depletion of host HSCs can be achieved without requiring immune suppression by radiation or chemotherapy, for example by administrating antibodies that disrupt HSC function and deplete HSCs along with other antibodies to make the host HSCs more vulnerable. For example, it has been shown that host HSC clearance is dependent on Fc-mediated antibody effector functions and that HSCs express CD47, a myeloid-specific immune checkpoint, which generates a “don't kill” signal and binds to SIRPα. See Chhabra, Science Translational Medicine, 8(351): 351ra105 (10 pages) (2016). Consequently, enhancing effector activity through blockade of CD47 extends anti-c-Kit conditioning to fully immunocompetent subjects. The treatment with c-Kit antibodies along with interruption of the CD47-SIRPα axis with CD47 antibodies leads to elimination of >99% of host HSCs and robust multilineage blood reconstitution after HSC transplantation. Consequently, in some aspects, anti-c-Kit monoclonal antibodies along with blockade of CD47 are used to clear subjects HSCs before transfusion of edited HSC.

In some aspects, cells that are directly involved in the body's immune response are harvested from the blood, including for example, lymphocytes (e.g., B cells, T cells and/or natural killer cells), monocytes and/or dendritic cells. Once harvested from the blood, these immune cells may be edited in vitro, optionally cultured, and then returned to the blood stream of the subject. Without being bound by theory, it is believed that many such cells have a half-life of approximately one year. Some methods for isolating these cells, according to the manufacturer instructions, include for example cell preparation tubes (CPT) containing sodium heparin manufactured by Becton Dickinson, FICOLL® Paque Premium (density of 1.077 g/mL) manufactured by GE Healthcare, and LYMPHOPREP™ using SEPMATE™ tubes, manufactured by STEMCELL Technologies. For strengths and weaknesses of different approaches in terms of cell isolation efficiency and cell viability, see for example Grievink, Biopreservation and Biobanking, 14(5): 410-415 (2016).

Cells encoding protective genetic variants and/or not encoding susceptibility genetic variants can be administered to the subject via any known methods (e.g., via venous administration or transplant).

Some aspects involve editing of the bone marrow stem cells that manufacture the cells of the blood. In some aspects, genetic modifying agents are delivered into bone marrow. In some aspects, the delivery includes passive targeting via polymers of neutral charge and suitable size (e.g., about 150 nm); liposomes surface-modified with an anionic glutamic acid for increased distribution into bone marrow via selective uptake by macrophages and greatly decreased distribution in liver and spleen; and/or molecules such as bisphosphates which bind specifically to bone-formation surfaces. See for example Chao-Feng, Biomaterials, 155: 191-202 (2017). These delivery mechanisms, which may for example include polymer wrappers, can be used to deliver CRISPR/Cas9 or other gene editing complexes, to the bone marrow.

Some aspects involve performing stem cell transplants directly into the bone marrow with the edited stem cells, either with or without radiation to reduce the population of bone marrow stem cells with the original germline alleles. Some aspects include methods where bone marrow stem cells are placed in the patient's blood stream (e.g., allowing the cells to find their way back to the bone marrow).

In some aspects, the subject in need of treatment is administered cells that have been obtained from the subject, and then genetically modified to decrease the amount of the susceptibility genetic variant and/or increase the amount of the protective genetic variant. In some aspects, the subject in need of treatment is administered cells obtained from a separate subject (e.g., cells from the separate subject that have genetically modified or that natively encode the desired genetic variants).

Some aspects comprise using gene editing in combination with other known therapeutics. For instance, exemplary HIV treatments include NRTIs (e.g., Abacavir, emtricitabine, lamivudine; Tenofovir disoproxil fumarate, or zidovudine), NNRTIs (e.g., Efavirenz, etravirine, nevirapine, or rilpivirine), fusion inhibitors (e.g., Enfuvirtide), protease inhibitors (e.g., Atazanavir, darunavir, fosamprenavir, ritonavir, saquinavir, or tipranavir), CCR5 Antagonists (e.g., Maraviroc), integrase inhibitors (e.g., Dolutegavir, raltegravir, elvitegravir, or bictegravir), post-attachment inhibitors (e.g., Ibalizumab), and pharmacokinetic enhancers (e.g., Cobicistat). HIV treatments are discussed in greater detail in Kemnic et al., “HIV Antiretroviral Therapy,” [Updated 2020 Jun. 23]. In: StatPearls [Internet], Treasure Island (FL): StatPearls Publishing; 2020 January, which is incorporated herein by reference in its entirety. Exemplary treatments for HSV include administration of nucleoside analogs (e.g., acyclovir, famciclovir, or valacyclovir). HSV treatments are discussed in more detail in Groves, “Genital Herpes: A Review,” American Family Physician, 93(11): 928-934 (2016), which is incorporated herein by reference in its entirety.

### Genetic Modifying Agents

Provided are methods and compositions that can be used to make desired genetic modifications, e.g., with the use of a genetic modifying agent. The genetic modifying agent may comprise a nuclease, such as CRISPR system, a zinc finger nuclease system, a TALEN, a meganuclease, or a RNAi system.

### CRISPR Systems

In some aspects, the genetic modifying agent is a CRISPR-Cas or CRISPR system, which refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” (e.g., RNA(s) to guide Cas, such as Cas9, e.g. CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). See, e.g., Shmakov, Molecular Cell, 60(3): 385-97 (2015); Zetsche, Cell, 163(3): P 759-771 (2015); WO 2014/093622 (PCT/US2013/074667).

In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise DNA polynucleotides or RNA polynucleotides. In some aspects, a target sequence is located in the nucleus of a cell. In some aspects, a target sequence is located in the cytoplasm of a cell.

In certain example aspects, the CRISPR effector protein may be delivered using a nucleic acid molecule encoding the CRISPR effector protein. The nucleic acid molecule encoding a CRISPR effector protein may advantageously be a codon optimized CRISPR effector protein (e.g., a sequence optimized for expression in eukaryote, e.g., humans (i.e. being optimized for expression in humans), or for another eukaryote, animal or mammal). See, e.g., WO 2014/093622 (PCT/US2013/074667).

Non-limiting examples of Cas proteins include Cas1, Cas1B, Cas2, Cas3, Cas4, Cas5, Cash, Cas7, Cas8, Cas9 (also known as Csn1 and Csx12), Cas10, Cpf1, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologues thereof, or modified versions thereof. While Cas9 is the most widely used Cas protein, other complexes can also be used to edit the DNA in possibly improved ways compared to Cas9. For example, Cpf1 implements a staggered cut in target DNA where there is an overhang on one strand of DNA enabling more specific DNA reassembly. Additionally, Cpf1 requires one CRISPR RNA (crRNA) for targeting while Cas9 requires both crRNA and transactivating crRNA (tracrRNA). The smaller crRNA enables multiplex genome editing since more of them can be packaged in a single vector. Additionally, whereas Cas9 cleaves the target DNA 3 nucleotide bases upstream, Cpf1 cleaves the target DNA 18-23 bases downstream from the target site, allowing the target region to remain intact and multiple rounds of cleavage at the target locus to increase the chance of a particular edit. This invention encompasses all the editing methods discussed here, as well as others, and is independent of the specific edit method used.

Some aspects involve vectors, e.g. for delivering or introducing in a cell Cas and/or RNA capable of guiding Cas to a target locus (i.e. guide RNA), but also for propagating these components. As used herein, a “vector” is a tool that allows or facilitates the transfer of an entity from one environment to another. It is a replicon, such as a plasmid, phage, or cosmid, into which another DNA segment may be inserted so as to bring about the replication of the inserted segment. Generally, a vector is capable of replication when associated with the proper control elements. In general, the term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g. circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g. retroviruses, replication defective retroviruses, adenoviruses, replication defective adenoviruses, and adeno-associated viruses (AAVs)). Viral vectors also include polynucleotides carried by a virus for transfection into a host cell. Certain vectors are capable of autonomous replication in a host cell into which they are introduced (e.g. bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids.

Recombinant expression vectors can comprise a nucleic acid in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. Within a recombinant expression vector, “operably linked” is intended to mean that the nucleotide sequence of interest is linked to the regulatory element(s) in a manner that allows for expression of the nucleotide sequence (e.g. in an in vitro transcription/translation system or in a host cell when the vector is introduced into the host cell).

The vector(s) can include the regulatory element(s), e.g., promoter(s). The vector(s) can comprise Cas encoding sequences, and/or a single, but possibly also can comprise at least 3 or 8 or 16 or 32 or 48 or 50 guide RNA(s) (e.g., sgRNAs) encoding sequences, such as 1-2, 1-3, 1-4 1-5, 3-6, 3-7, 3-8, 3-9, 3-10, 3-8, 3-16, 3-30, 3-32, 3-48, 3-50 RNA(s) (e.g., sgRNAs). In a single vector there can be a promoter for each RNA (e.g., sgRNA), advantageously when there are up to about 16 RNA(s); and, when a single vector provides for more than 16 RNA(s), one or more promoter(s) can drive expression of more than one of the RNA(s), e.g., when there are 32 RNA(s), each promoter can drive expression of two RNA(s), and when there are 48 RNA(s), each promoter can drive expression of three RNA(s).

### Guide Molecules

The term “guide sequence” and “guide molecule” encompasses any polynucleotide sequence having sufficient complementarity with a target nucleic acid sequence to hybridize with the target nucleic acid sequence and direct sequence-specific binding of a nucleic acid-targeting complex to the target nucleic acid sequence. The guide sequences made using the methods disclosed herein may be a full-length guide sequence, a truncated guide sequence, a full-length sgRNA sequence, a truncated sgRNA sequence, or an E+F sgRNA sequence. While sgRNA is meant to refer to “single guide RNA,” it should be understood that the same could be achieved with two separate RNA molecules, one customizable crispr RNA (crRNA) which hold the complementary sequence to the DNA to the target DNA, and the tracr RNA which serves as a binding scaffold for the Cas nuclease. In some aspects, the degree of complementarity of the guide sequence to a given target sequence, when optimally aligned using a suitable alignment algorithm, is about or more than about 50%, 60%, 75%, 80%, 85%, 90%, 95%, 97.5%, 99%, or more. In certain example aspects, the guide molecule comprises a guide sequence that may be designed to have at least one mismatch with the target sequence, such that a RNA duplex is formed between the guide sequence and the target sequence. In some aspects, the guide sequence is designed to have a stretch of two or more adjacent mismatching nucleotides, such that the degree of complementarity over the entire guide sequence is further reduced. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies), ELAND (Illumina, San Diego, Calif.), SOAP, and Maq. The ability of a guide sequence (within a nucleic acid-targeting guide RNA) to direct sequence-specific binding of a nucleic acid-targeting complex to a target nucleic acid sequence may be assessed by any suitable assay. For example, the components of a nucleic acid-targeting CRISPR system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay as described herein. Similarly, cleavage of a target nucleic acid sequence (or a sequence in the vicinity thereof) may be evaluated in a test tube by providing the target nucleic acid sequence, components of a nucleic acid-targeting complex, including the guide sequence to be tested and a control guide sequence different from the test guide sequence, and comparing binding or rate of cleavage at or in the vicinity of the target sequence between the test and control guide sequence reactions. A guide sequence, and hence a nucleic acid-targeting guide RNA may be selected to target any target nucleic acid sequence.

In certain aspects, the guide sequence or spacer length of the guide molecules is from 15 to 50 nucleotides (nt). In certain aspects, the spacer length of the guide RNA is at least 15 nucleotides. In certain aspects, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27-30 nt, e.g., 27, 28, 29, or 30 nt, from 30-35 nt, e.g., 30, 31, 32, 33, 34, or 35 nt, or 35 nt or longer. In certain example aspect, the guide sequence is 15, 16, 17, 18, 19, 20, 21, 22, 23, 24, 25, 26, 27, 28, 29, 30, 31, 32, 33, 34, 35, 36, 37, 38, 39 40, 41, 42, 43, 44, 45, 46, 47 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nt.

In certain aspects, the guide molecule comprises (1) a guide sequence capable of hybridizing to a target locus and (2) a tracr mate or direct repeat sequence whereby the direct repeat sequence is located upstream (i.e., 5′) from the guide sequence. In some aspects, the seed sequence (i.e. the sequence involved with recognition and/or hybridization to the sequence at the target locus) of the guide sequence is approximately within the first 10 nucleotides of the guide sequence.

In a particular aspect the guide molecule comprises a guide sequence linked to a direct repeat sequence, wherein the direct repeat sequence comprises one or more stem loops or optimized secondary structures. In particular aspects, the direct repeat has a minimum length of 16 nts and a single stem loop. In further aspects the direct repeat has a length longer than 16 nts, preferably more than 17 nts, and has more than one stem loops or optimized secondary structures. In particular aspects the guide molecule comprises or consists of the guide sequence linked to all or part of the natural direct repeat sequence. A typical Type V or Type VI CRISPR-cas guide molecule comprises (in 3′ to 5′ direction or in 5′ to 3′ direction): a guide sequence, a first complimentary stretch (the “repeat”), a loop (which is typically 4 or 5 nucleotides long), a second complimentary stretch (the “anti-repeat” being complimentary to the repeat), and a poly A (often poly U in RNA) tail (terminator). In certain aspects, the direct repeat sequence retains its natural architecture and forms a single stem loop. In particular aspects, certain aspects of the guide architecture can be modified, for example by addition, subtraction, or substitution of features, whereas certain other aspects of guide architecture are maintained. Preferred locations for engineered guide molecule modifications, including but not limited to insertions, deletions, and substitutions include guide termini and regions of the guide molecule that are exposed when complexed with the CRISPR-Cas protein and/or target, for example the stemloop of the direct repeat sequence.

In some aspects, the CRISPR system effector protein is an RNA-targeting effector protein. In certain aspects, the CRISPR system effector protein is a Type VI CRISPR system targeting RNA (e.g., Cas13a, Cas13b, Cas13c or Cas13d). Example RNA-targeting effector proteins include Cas13b and C2c2 (also known as Cas13a). As used herein, the term “Cas13” refers to any Type VI CRISPR system targeting RNA (e.g., Cas13a, Cas13b, Cas13c or Cas13d). When the CRISPR protein is a C2c2 protein, a tracrRNA is not required. C2c2 or Cas13 have been described in Abudayyeh et al., Science, 353(6299): aaf5573-1-aaf5573-9 (2016); Shmakov, Molecular Cell, 60(3): 385-97 (2015); and Smargon, Molecular Cell, 65: 618-30 (2017).

In some aspects, one or more elements of a nucleic acid-targeting system is derived from a particular organism comprising an endogenous CRISPR RNA-targeting system. In certain example aspects, the effector protein CRISPR RNA-targeting system comprises at least one HEPN domain. In certain example aspects, the effector protein comprises a single HEPN domain. In certain other example aspects, the effector protein comprises two HEPN domains.

In certain other example aspects, the CRISPR system effector protein is a C2c2 nuclease. The activity of C2c2 may depend on the presence of two HEPN domains. These have been shown to be RNase domains, i.e. nuclease (in particular an endonuclease) cutting RNA. C2c2 HEPN may also target DNA, or potentially DNA and/or RNA. On the basis that the HEPN domains of C2c2 are at least capable of binding to and, in their wild-type form, cutting RNA, then it is preferred that the C2c2 effector protein has RNase function. See Abudayyeh, Science, 353(6299): aaf5573-1-aaf5573-9 (2016).

### Tale Systems

In some aspects, genetic modification is made by way of the transcription activator-like effector nucleases (TALENs) system. Transcription activator-like effectors (TALEs) can be engineered to bind practically any desired DNA sequence. Exemplary methods of genome editing using the TALEN system can be found for example in Cermak, Nucleic Acids Res., 39(21):e82 (2011); Zhang, Nat. Biotechnol., 29: 149-153 (2011) and U.S. Pat. Nos. 8,450,471, 8,440,431 and 8,440,432.

Some aspects comprise using isolated, non-naturally occurring, recombinant or engineered DNA binding proteins that comprise TALE monomers as a part of their organizational structure that enable the targeting of nucleic acid sequences with improved efficiency and expanded specificity. The structure and function of TALEs is further described in, for example, Moscou, Science, 326: 1501 (2009); Boch, Science, 326: 1509-1512 (2009); and Zhang, Nat. Biotechnology, 29: 149-153 (2011).

### ZN-Finger Nucleases

In some aspects, the genetic modifying agent includes a zinc finger system. One type of programmable DNA-binding domain is provided by artificial zinc-finger (ZF) technology, which involves arrays of ZF modules to target new DNA-binding sites in the genome. Each finger module in a ZF array targets three DNA bases. A customized array of individual zinc finger domains is assembled into a ZF protein (ZFP).

ZFPs can comprise a functional domain. The first synthetic zinc finger nucleases (ZFNs) were developed by fusing a ZF protein to the catalytic domain of the Type IIS restriction enzyme Fokl. (Kim, Proc. Natl. Acad. Sci. U.S.A., 91, 883-887 (1994); Kim, Proc. Natl. Acad. Sci. U.S.A., 93, 1156-1160 (1996)). Increased cleavage specificity can be attained with decreased off target activity by use of paired ZFN heterodimers, each targeting different nucleotide sequences separated by a short spacer. (Doyon, Nat. Methods, 8: 74-79 (2011)). ZFPs can also be designed as transcription activators and repressors and have been used to target many genes in a wide variety of organisms. Exemplary methods of genome editing using ZFNs can be found for example in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626.

### Meganucleases

As disclosed herein editing can be made by way of meganucleases, which are endodeoxyribonucleases characterized by a large recognition site (double-stranded DNA sequences of 12 to 40 base pairs). Exemplary methods for using meganucleases can be found in U.S. Pat. Nos. 8,163,514; 8,133,697; 8,021,867; 8,119,361; 8,119,381; 8,124,369; and 8,129,134.

### RNAi

In certain aspects, the genetic modifying agent is RNAi (e.g., shRNA). As used herein, “gene silencing” or “gene silenced” in reference to an activity of an RNAi molecule, for example a siRNA or miRNA refers to a decrease in the mRNA level in a cell for a target gene by at least about 5%, about 10%, about 20%, about 30%, about 40%, about 50%, about 60%, about 70%, about 80%, about 90%, about 95%, about 99%, about 100% of the mRNA level found in the cell without the presence of the miRNA or RNA interference molecule. In one preferred aspect, the mRNA levels are decreased by at least about 70%, about 80%, about 90%, about 95%, about 99%, about 100%.

As used herein, the term “RNAi” refers to any type of interfering RNA, including but not limited to, siRNAi, shRNAi, endogenous microRNA and artificial microRNA. For instance, it includes sequences previously identified as siRNA, regardless of the mechanism of down-stream processing of the RNA (i.e. although siRNAs are believed to have a specific method of in vivo processing resulting in the cleavage of mRNA, such sequences can be incorporated into the vectors in the context of the flanking sequences described herein). The term “RNAi” can include both gene silencing RNAi molecules, and also RNAi effector molecules which activate the expression of a gene.

As used herein, a “siRNA” refers to a nucleic acid that forms a double stranded RNA, which double stranded RNA has the ability to reduce or inhibit expression of a gene or target gene when the siRNA is present or expressed in the same cell as the target gene. The double stranded RNA siRNA can be formed by the complementary strands. In one aspect, a siRNA refers to a nucleic acid that can form a double stranded siRNA. The sequence of the siRNA can correspond to the full-length target gene, or a subsequence thereof. Typically, the siRNA is at least about 15-50 nucleotides in length (e.g., each complementary sequence of the double stranded siRNA is about 15-50 nucleotides in length, and the double stranded siRNA is about 15-50 base pairs in length, preferably about 19-30 base nucleotides, preferably about 20-25 nucleotides in length, e.g., 20, 21, 22, 23, 24, 25, 26, 27, 28, 29, or 30 nucleotides in length).

As used herein “shRNA” or “small hairpin RNA” (also called stem loop) is a type of siRNA. In one aspect, these shRNAs are composed of a short, e.g. about 19 to about 25 nucleotides, antisense strand, followed by a nucleotide loop of about 5 to about 9 nucleotides, and the analogous sense strand. Alternatively, the sense strand can precede the nucleotide loop structure and the antisense strand can follow.

The terms “microRNA” or “miRNA” are used interchangeably herein are endogenous RNAs, some of which are known to regulate the expression of protein-coding genes at the posttranscriptional level. Endogenous microRNAs are small RNAs naturally present in the genome that are capable of modulating the productive utilization of mRNA. The term artificial microRNA includes any type of RNA sequence, other than endogenous microRNA, which is capable of modulating the productive utilization of mRNA. MicroRNA sequences have been described in publications such as Lim, Genes & Development, 17: 991-1008 (2003), Lim, Science, 299, 1540 (2003), Lee and Ambros, Science, 294, 862 (2001), Lau, Science, 294, 858-861 (2001), Lagos-Quintana, Current Biology, 12: 735-739 (2002), Lagos Quintana, Science, 294, 853-857 (2001), and Lagos-Quintana, RNA, 9: 175-179 (2003). Multiple microRNAs can also be incorporated into a precursor molecule. Furthermore, miRNA-like stem-loops can be expressed in cells as a vehicle to deliver artificial miRNAs and short interfering RNAs (siRNAs) for the purpose of modulating the expression of endogenous genes through the miRNA and or RNAi pathways.

As used herein, “double stranded RNA” or “dsRNA” refers to RNA molecules that are comprised of two strands. Double-stranded molecules include those comprised of a single RNA molecule that doubles back on itself to form a two-stranded structure. For example, the stem loop structure of the progenitor molecules from which the single-stranded miRNA is derived, called the pre-miRNA (Bartel, Cell, 116(2): 281-297 (2004)), comprises a dsRNA molecule.

Some aspects involve methods of generating a eukaryotic cell comprising a modified or edited gene. In some aspects, the method comprises (a) introducing one or more vectors into a eukaryotic cell, wherein the one or more vectors drive expression of one or more of: Cas effector module, and a guide sequence linked to a direct repeat sequence, wherein the Cas effector module associate one or more effector domains that mediate base editing, and (b) allowing a CRISPR-Cas effector module complex to bind to a target polynucleotide to effect base editing of the target polynucleotide within said disease gene, wherein the CRISPR-Cas effector module complex comprises a Cas effector module complexed with the guide sequence that is hybridized to the target sequence within the target polynucleotide.

A further aspect relates to an isolated cell obtained or obtainable from the methods described herein comprising the composition described herein or progeny of said modified cell. In particular aspects, the cell is a eukaryotic cell, preferably a human or non-human animal cell. In some aspects the cell is an immune cell. In some aspects, the cell is a HSC.

Some aspects comprise isolated immune cells or hematopoietic stem cells, wherein the cellular DNA has been modified via gene editing to (a) reduce the amount of one or more genetic variants associated with susceptibility to a viral infection; and/or (b) increase the amount of one or more genetic variants protective against the viral infection. Some aspects comprise a population of immune cells or hematopoietic stem cells, wherein at least about 10% of the cells in the population have been modified via gene editing to (a) reduce the amount of one or more genetic variants associated with susceptibility to a viral infection; and/or (b) increase the amount of one or more genetic variants protective against the viral infection.

The invention further relates to a method for cell therapy, comprising administering to a patient in need thereof the modified cell described herein, wherein the presence of the modified cell remedies a disease in the patient.

What is claimed is:

1. A method of treating a viral infection, the method comprising administering, to a subject infected with a virus, a genetic modifying agent that targets one or more regions of the virus genome that are maximally different from the subject's genome.

2. The method of claim 1, wherein the targeted region of the virus genome has less than 50% sequence homology with a region of the subject's genome, less than 40% sequence homology with a region of the subject's genome, less than 30% sequence homology with a region of the subject's genome, less than 20% sequence homology with a region of the subject's genome, less than 10% sequence homology with a region of the subject's genome, less than 5% sequence homology with a region of the subject's genome, or less than 1% sequence homology with a region of the subject's genome.

3. The method of claim 1 or 2, wherein the genetic modifying agent is formulated in a carrier that has an increased affinity for cell types or tissue types harboring the viral infection as compared to cell types or tissue types not harboring the viral infection.

4. The method of claim 3, wherein the carrier comprises an adeno-associated virus vector, a lentiviral vector, or a polymer nanoparticle.

5. The method of claim 3 or 4, wherein the carrier comprises a polymer nanoparticle that comprises one or more of a cholesterol, a lipid-poly(ethylene glycol) (lipid-PEG) compound, an ionizable cationic lipid, a distearoylphosphatidylcholine, and a helper lipid (e.g., dioleoyl phosphatidylethanolamine (DOPE) and/or dioleoylphosphatidylcholine (DOPC)).

6. A method of treating a viral infection, the method comprising administering, to a subject infected with a virus, a genetic modifying agent that targets one or more genes of host cells that harbor the virus, where the targeted gene(s) are necessary to the correct function and replication of the virus but are dispensable to the host cells.

7. A method of treating a viral infection, the method comprising
(a) decreasing, in one or more cells in the subject, the amount of one or more genetic variants associated with susceptibility to the viral infection (“susceptibility genetic variant(s)”); and/or
(b) increasing, in one or more cells in the subject, the amount of one or more genetic variants protective against the viral infection (“protective genetic variant(s)”).

8. The method of claim 7, comprising decreasing the amount of the susceptibility genetic variant in one or more immune cells and/or one or more hematopoietic stem cells in the subject, and/or
increasing the amount of the protective genetic variant in one or more immune cells and/or one or more hematopoietic stem cells in the subject.

9. The method of claim 8, wherein the immune cells comprise one or more of leukocytes, phagocytes, macrophages, neutrophils, dendritic cells, innate lymphoid cells, eosinophils, basophils, natural killer cells, B cells, and T cells.

10. The method of any one of claims 7-9, comprising administering to the subject:
immune cells and/or hematopoietic stem cells containing the protective genetic variant; and/or
immune cells and/or hematopoietic stem cells that contain the protective genetic variant and do not contain the susceptibility genetic variant.

11. The method of any one of claims 7-9, comprising administering to the subject (i) a genetic modifying agent that decreases the amount of the susceptibility genetic variant in one or more immune cells and/or one or more hematopoietic stem cells in the subject, and/or (ii) a genetic modifying agent that increases the amount of the protective genetic variant in one or more immune cells and/or one or more hematopoietic stem cells in the subject.

12. The method of any one of claims 7-11, wherein a proportion of protective protein variants:susceptibility protein variants in the subject is increased.

13. The method of any one of claims 1-6 and 11-12, wherein the genetic modifying agent comprises a nuclease.

14. The method of claim 13, wherein the nuclease is (1) a class 2 clustered regularly-interspaced short palindromic repeat (CRISPR) associated nuclease, (2) a zinc finger nuclease (ZFN), (3) a Transcription Activator-Like Effector nuclease (TALEN), or (4) a meganuclease.

15. The method of claim 13 or 14, wherein the nuclease comprises Cas1, Cas1B, Cas2, Cas3, Cas4, Cas5, Cash, Cas7, Cas8, Cas9, Cas10, Cpf1, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, or Csf4.

16. The method of any one of claims 1-6 and 11-15, wherein the genetic modifying agent comprises a Cas9 protein and a guide RNA.

17. The method of any one of claims 1-6 and 11-15, wherein the genetic modifying agent comprises one or both of CRISRP-Cas9 and a guide RNA.

18. The method of any one of claims 1-6 and 11-17, wherein the genetic modifying agent is activated and/or deactivated using a chemical, biological, or optical input.

19. A method of identifying viral genes associated with survival of a virus, the method comprising editing one or more viral genes and assessing the ability of the virus to replicate and survive with the edited genes.

20. The method of claim 19, comprising editing two, three, or more viral genes.

21. The method of claim 19 or 20, wherein the one or more viral genes are maximally different from a host genome.

22. The method of any one of claims 1-21, wherein the virus comprises HIV, HSV-1, or HSV-2.
